(19) United States
(12) Patent Application Publication
(10) Pub. No.: US 2015/0119760 A1
(43) Pub. Date: Apr. 30, 2015

(54) SYSTEM AND METHOD FOR PROVIDING A SMART ACTIVITY SCORE

(71) Applicant: JayBird LLC, Salt Lake City, UT (US)

(72) Inventors: BEN WISBEY, Salt Lake City, UT (US); DAVID SHEPHERD, Salt Lake City, UT (US); HAGEN DIESTERBECK, Salt Lake City, UT (US); JUDD ARMSTRONG, Salt Lake City, UT (US)

(73) Assignee: JAYBIRD LLC, Salt Lake City, UT (US)

(21) Appl. No.: 14/137,734

(22) Filed: Dec. 20, 2013

Related U.S. Application Data
(63) Continuation-in-part of application No. 14/062,815, filed on Oct. 24, 2013.

(51) Int. Cl. A61B 5/11; A61B 5/00

(52) U.S. Cl. CPC A61B 5/1118; A61B 5/4866
USPC 600/595

(57) ABSTRACT

A system for providing a smart activity score includes an apparatus for providing a smart activity score. The apparatus includes a movement monitoring module that monitors a movement to determine a metabolic loading associated with the movement during a score period. The apparatus also includes a period activity score module that creates and updates a period activity score based on the metabolic loading and the movement. The period activity score is created and updated for the score period. In addition, the apparatus includes a smart activity score module that creates and updates a smart activity score by aggregating a set of period activity scores.

### CROSS-REFERENCE TO RELATED APPLICATIONS

This application is a continuation-in-part of and claims the benefit of U.S. patent application Ser. No. 14/062,815, filed Oct. 24, 2013, titled “Wristband with Removable Activity Monitoring Device,” the contents of which are incorporated herein by reference.

### TECHNICAL FIELD

The present disclosure relates generally to fitness monitoring devices, and more particularly to systems and methods for providing a smart activity score.

### DESCRIPTION OF THE RELATED ART

Previous generation movement monitoring and fitness tracking devices generally enabled only a tracking of activity that accounts for estimated total calories burned. Currently available fitness tracking devices now add functionality that use universal metabolic equivalent tasks to track activity and performance. Issues with currently available fitness tracking devices include that they do not track user activities at a granular level and do not tightly couple metabolic equivalents to user characteristics. Another issue is that currently available solutions do not account in a precise manner for the health and performance benefits of sustained activity.

### BRIEF SUMMARY OF THE DISCLOSURE

In view of the above drawbacks, there exists a long-felt need for fitness monitoring devices that track user activities at a granular level and that are tightly coupled to user characteristics. Further, there is a need for fitness monitoring devices that provide increased resolution into the performance benefits of sustained activity.

Embodiments of the present disclosure provide systems and methods for providing a smart activity score.

One embodiment involves an apparatus for providing a smart activity score. The apparatus includes a movement monitoring module that monitors a movement to determine a metabolic loading associated with the movement. The movement monitoring module monitors the movement during a score period. The apparatus further includes a period activity score module that creates and updates a period activity score based on the metabolic loading and the movement. The period activity score is created and updated for the score period. In one embodiment, the score period is ten seconds. The apparatus also includes a smart activity score module that creates and updates a smart activity score by aggregating a set of period activity scores.

In one embodiment, the metabolic loading is determined from a set of metabolic loadings, each metabolic loading being determined according to user information from a user. The smart activity score is associated with a measuring period. In such embodiments, the smart activity score module calculates an average smart activity score from a set of past smart activity score. Each past smart activity score is associated with a past measuring period. The user information includes a user lifestyle selected from a set of reference lifestyles.

The apparatus for providing a smart activity score, in one embodiment, includes a user lifestyle module. The user lifestyle module associates each reference lifestyle with a lower threshold score and an upper threshold score. The lower threshold score and the upper threshold score associated with each reference lifestyle define a range or scores. No two ranges of scores overlap. The user lifestyle module changes the user lifestyle to the reference lifestyle associated with the lower threshold score not greater than the average smart activity score and the upper threshold score not less than the average smart activity score.

In one embodiment, the apparatus includes a period activity score multiplier module that applies a period activity score multiplier to the period activity score to create an adjusted period activity score. In such an embodiment, the smart activity score includes an aggregation of adjusted period activity scores. The period activity score multiplier, in one embodiment, is directly proportional to the number of continuous score periods over which a user activity type of the movement and a user activity intensity of the movement is maintained. In a further embodiment, the period activity score multiplier is directly proportional to the smart activity score for the current measuring period. In various embodiments, at least one of the movement monitoring module, the period activity score module, and the smart activity score module is embodied in a sensor configured to be attached to the body of a user.

The disclosure, in one embodiment, involves a method for providing a smart activity score. The method includes monitoring a movement to determine a metabolic loading associated with the movement. The movement is monitored during a score period. The score period, in one embodiment, is ten seconds. The method also includes creating and updating a period activity score based on the metabolic loading and the movement. The period activity score is created and updated for the score period. The method further includes creating and updating a smart activity score by aggregating a set of period activity scores.

In one embodiment, the metabolic loading is determined from a set of metabolic loadings and each metabolic loading is determined according to user information from a user. The user information includes a user lifestyle selected from a set of reference lifestyles. Determining the set of metabolic loadings is based on the user lifestyle. The method, in one embodiment, includes associating each reference lifestyle with a lower threshold score and an upper threshold score. The lower threshold score and the upper threshold score associated with each reference lifestyle define a range of scores. No two ranges of scores overlap. In one embodiment, the method includes calculating an average smart activity score from a set of past smart activity scores. Each past smart activity score is associated with a past measuring period. In one embodiment, the method includes changing the user lifestyle to the reference lifestyle associated with the lower threshold score not greater than the average smart activity score and the upper threshold score not less than the average smart activity score.

In one embodiment, the method includes comparing the smart activity score to a past smart activity score. The smart activity score is associated with a measuring period. The past smart activity score is associated with a past measuring period. The method may include receiving a second smart activity score from a second user. In one embodiment, the method includes comparing the smart activity score to the second smart activity score.

The method for providing the smart activity score, in one embodiment, includes applying a score period multiplier to the period activity score to create an adjusted period activity score. The smart activity score, in such an embodiment, includes an aggregation of adjusted period activity scores. In one embodiment, the score period multiplier is directly proportional to the number of continuous score periods over which a user activity type of the movement and a user activity intensity of the movement is maintained. The score period multiplier, in a further embodiment, is directly proportional to the smart activity score for the current measuring period. In various embodiments, at least one of the operations of monitoring the movement, creating and updating the period activity score, and creating and updating the smart activity score is accomplished using a sensor configured to be attached to the body of the user.

One embodiment includes a system for providing a smart activity score. The system includes a processor and at least one computer program residing on the processor. The computer program is stored on a non-transitory computer readable medium having computer executable program code embodied thereon. The computer executable program code is configured to monitor a movement to determine a metabolic loading associated with the movement during a score period. The computer executable program code is further configured to create and update a period activity score based on the metabolic loading and the movement during the score period. The computer executable program code is configured to create and update a smart activity score by aggregating a set of period activity scores.

Other features and aspects of the disclosure will become apparent from the following detailed description, taken in conjunction with the accompanying drawings, which illustrate, by way of example, the features in accordance with embodiments of the disclosure. The summary is not intended to limit the scope of the disclosure, which is defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments of the disclosure.

FIG. 1 illustrates a cross-sectional view of the wristband and electronic modules of an example activity monitoring device.

FIG. 2 illustrates a perspective view of an example activity monitoring device.

FIG. 3 illustrates a cross-sectional view of an example assembled activity monitoring device.

FIG. 4 illustrates a side view of an example electronic capsule.

FIG. 5 illustrates a cross-sectional view of an example electronic capsule.

FIG. 6 illustrates perspective views of wristbands as used in one embodiment of the disclosed activity monitoring device.

FIG. 7 illustrates an example system for providing a smart activity score.

FIG. 8 illustrates an example apparatus for providing a smart activity score.

FIG. 9 illustrates another example apparatus for providing a smart activity score.

FIG. 10A is an operational flow diagram illustrating an example of a method for creating and updating a smart activity score.

FIG. 10B is an example of a metabolic loading table.

FIG. 10C is an example of an activity intensity library.

FIG. 11 is an operational flow diagram illustrating an example of a method for creating and updating a smart activity score including basing the smart activity score on a user lifestyle and changing the user lifestyle.

FIG. 12 is an operational flow diagram illustrating an example of a method for creating and updating a smart activity score including comparing the smart activity score to other smart activity scores.

FIG. 13 illustrates an example computing module that may be used to implement various features of the systems and methods disclosed herein.

The figures are not intended to be exhaustive or to limit the disclosure to the precise form disclosed. It should be understood that the disclosure can be practiced with modification and alteration, and that the disclosure can be limited only by the claims and the equivalents thereof.

### DETAILED DESCRIPTION

Embodiments of the present disclosure are directed toward systems and methods for providing a smart activity score.

The disclosure is directed toward various embodiments of such systems and methods. In one such embodiment, the systems and methods are directed to a device that provides a smart activity score. According to some embodiments of the disclosure, the device may be an electronic capsule embedded in and removable from an attachable device that may be attached to a user. In one embodiment, the attachable device is a wristband that includes an activity monitoring device.

FIG. 1 is a diagram illustrating a cross-sectional view of an exemplary embodiment of an activity monitoring device. Referring now to FIG. 1, an activity monitoring device comprises an electronic capsule 200 and a wristband 100. Electronic capsule 200 comprises wrist biosensor 210, finger biosensor 220, battery 230, one or more logic circuits 240, and casing 250.

In some embodiments, one or more logic circuits 240 include an accelerometer, a wireless transmitter, and circuitry. Logic circuits 240 may further include a gyroscope. Logic circuits 240 may be configured to process electronic input signals from the biosensors and the accelerometer, store the processed signals as data, and output the data using the wireless transmitter. The transmitter is configured to communicate using available wireless communications standards. For example, in some embodiments, the wireless transmitter may be a BLUETOOTH transmitter, a Wi-Fi transmitter, a GPS transmitter, a cellular transmitter, or some combination thereof. In an alternative embodiment, the wireless transmitter may further comprise a wired interface (e.g. USB, fiber optic, HDMI, etc.) for communicating stored data.

Logic circuits 240 are electrically coupled to wrist biosensor 210 and finger biosensor 220. In addition, logic circuits 240 are configured to receive and process a plurality of electric signals from each of wrist biosensor 210 and finger biosensor 220. In some embodiments, the plurality of electric signals comprise an activation time signal and a recovery time signal such that logic circuits 240 may process the plurality of signals to calculate an activation recovery interval equal to the difference between the activation time signal and the recovery time signal. In some embodiments, the plurality of signals may comprise electro-cardio signals from a heart, and the logic circuits may process the electro-cardio signals to calculate and store a RR-interval, and the RR-interval may be used to calculate and store a heart rate variability (HRV) value. Here, the RR-interval is equal to the delta in time between two R-waves, where the R-waves are the electro-cardio signals generated by a ventricle contraction in the heart.

In some embodiments, logic circuits 240 detect and store metrics such as the amount of physical activity, sleep, or rest over a recent period of time, or the amount of time without physical activity over a recent period of time. Logic circuits 240 may then use the HRV, or the HRV in combination with said metrics, to calculate a recovery score. For example, logic circuits 240 may detect the amount of physical activity and the amount of sleep a user experienced over the last 48 hours, combine those metrics with the user's HRV, and calculate a recovery score of between 1 and 10, wherein the recovery score may indicate the user's physical condition and aptitude for further physical activity that day. The recovery score may also be calculated on a scale of between 1 and 100, or any other scale or range.

In some embodiments, finger biosensor 220 and wrist biosensor 240 are replaced or supplemented by a single biosensor. In one such embodiment, the single biosensor is an optical biosensor such as a pulse oximeter configured to detect blood oxygen saturation levels. The pulse oximeter may then output a signal to the logic circuits indicating detected a cardiac cycle phase, and logic circuits 240 may use cardiac cycle phase data to calculate a HRV value.

Wristband 100 comprises material 110 configured to encircle a human wrist. In one embodiment, wristband 100 is adjustable. Cavity 120 is notched on the radially inward facing side of the wristband and shaped to substantially the same dimensions as the profile of the electronic capsule. In addition, aperture 130 is located in material 110 within cavity 120. Aperture 130 is shaped to substantially the same dimensions as the profile of finger biosensor 220. The combination of cavity 120 and aperture 130 is designed to detachably couple to electric capsule 200 such that, when electric capsule 200 is positioned inside cavity 120, finger biosensor 220 protrudes through aperture 130. Electronic capsule 200 may further comprise one or more magnets 260 configured to secure capsule 200 to cavity 120. Magnets 260 may be concealed in casing 250. In addition, cavity 120 may be configured to conceal magnets 260 when electric capsule 200 detachably couples to the cavity and aperture combination.

Wristband 100 may further comprise steel strip 140 concealed in material 110 within cavity 120. In this embodiment, when the electronic capsule 200 is positioned within cavity 120, one or more magnets 260 are attracted to steel strip 140 and pull electronic capsule 200 radially outward with respect to wristband 100. The force provided by magnets 260 may detachably secure electronic capsule 200 inside cavity 120. In alternative embodiments, electronic capsule 200 is positioned inside cavity 120 and affixed using a form-fit, press-fit, snap-fit, friction-fit, VELCRO, or other temporary adhesion or attachment technology.

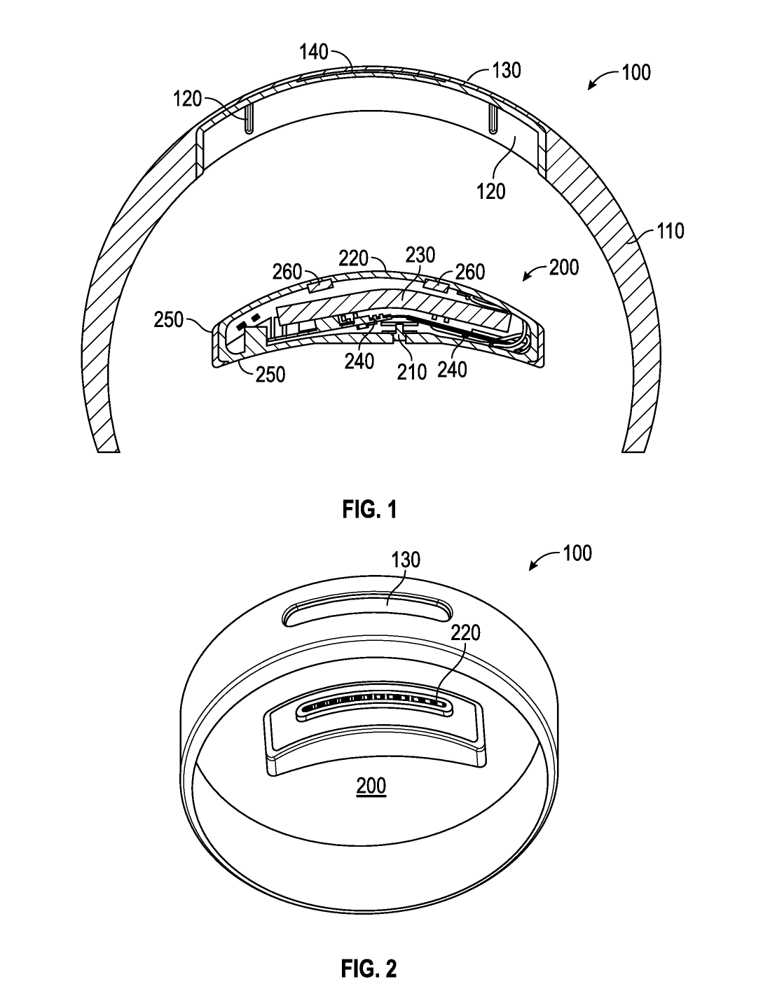

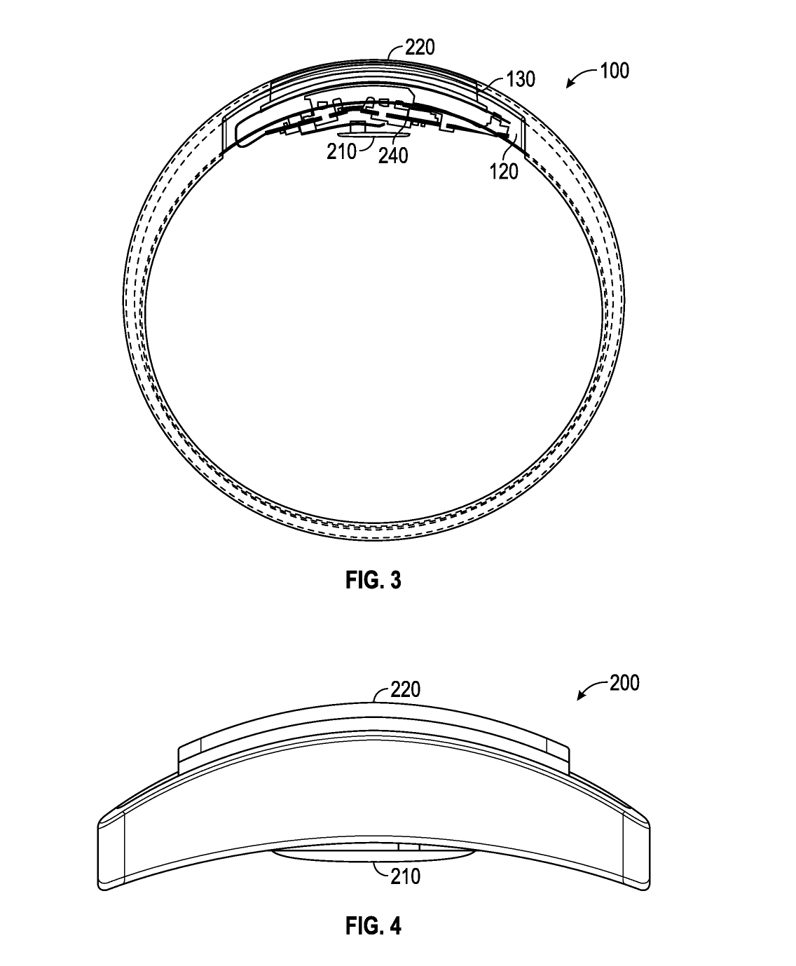

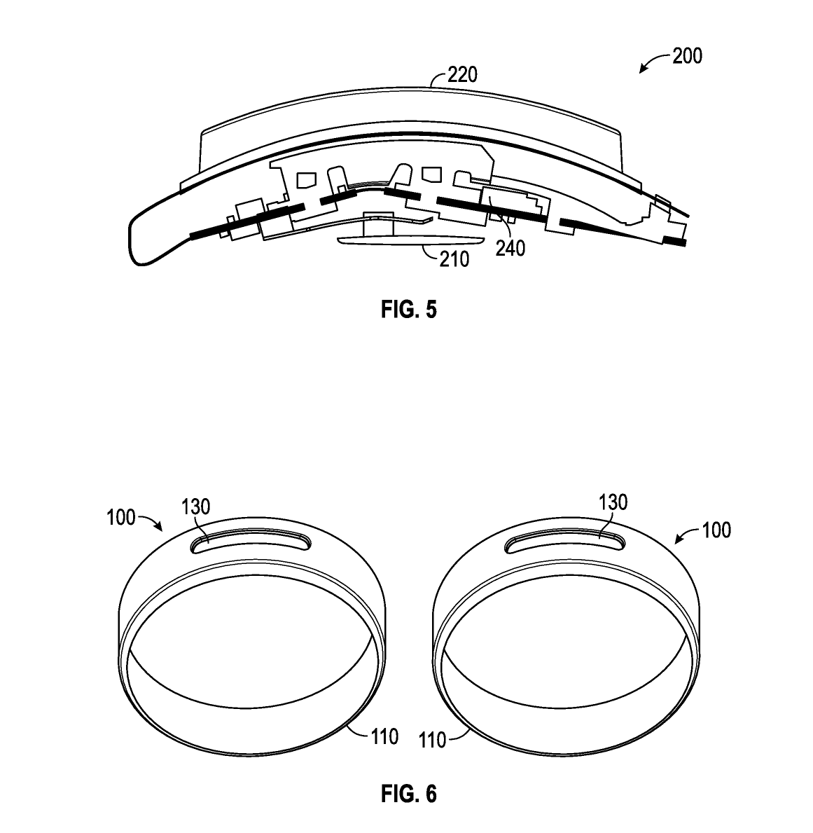

FIG. 2 illustrates a perspective view of one embodiment of the disclosed activity monitoring device, in which wristband 100 and electronic capsule 200 are unassembled. FIG. 3 illustrates a cross-sectional view of one embodiment of a fully assembled wristband 100 with removable athletic monitoring device. FIG. 4 illustrates a side view of an electronic capsule 200 according to one embodiment of the disclosure. FIG. 5 illustrates a cross-sectional view of electronic capsule 200. FIG. 6 is a perspective view of two possible variants of the wristband according to some embodiments of the disclosure. Wristbands 100 may be constructed with different dimensions, including different diameters, widths, and thicknesses, in order to accommodate different human wrist sizes and different preferences.

In some embodiments of the disclosure, electronic capsule 200 may be detachably coupled to a cavity on a shoe and/or a sock. In other embodiments, electronic capsule 200 is detachably coupled to sports equipment. For example, electronic capsule 200 may be detachably coupled to a skateboard, a bicycle, a helmet, a surfboard, a paddle boat, a body board, a hang glider, or other piece of sports equipment. In these embodiments, electronic capsule is affixed to the sports equipment using magnets. Alternatively, in other embodiments, electronic capsule 200 is affixed using a form-fit, snap-fit, press-fit, friction-fit suction cup, VELCRO, or other technology that would be apparent to one of ordinary skill in the art.

In one embodiment of the disclosure, electronic capsule 200 includes an optical sensor such as a heart rate sensor or oximeter. In this embodiment, the optical sensor is positioned to face radially inward towards a human wrist when wristband 100 is fit on the human wrist. Alternatively, the optical sensor may be separate from electronic capsule 200, but still detachably coupled to wristband 100 and electronically coupled to the circuit boards enclosed in electronic capsule 200. Wristband 100 and electronic capsule 200 may operate in conjunction with a system for providing a smart activity score.

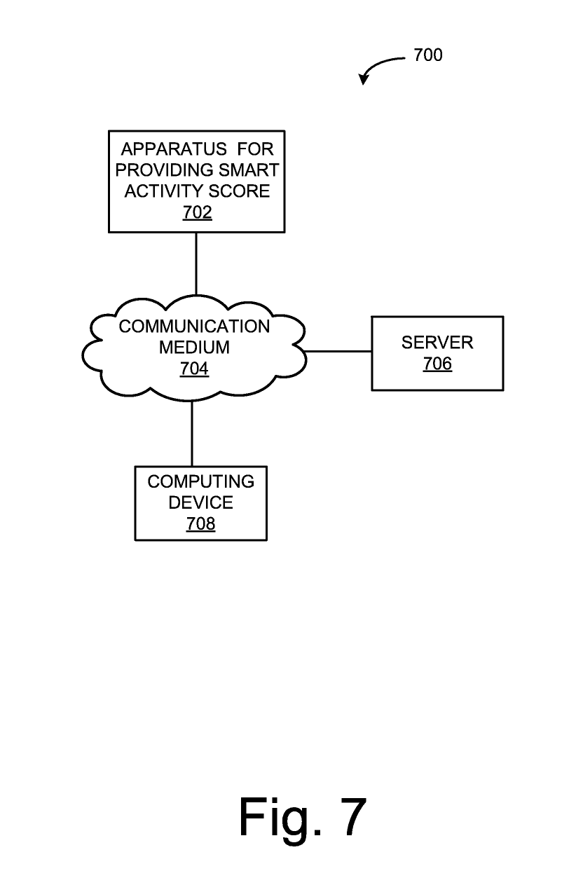

FIG. 7 is a schematic block diagram illustrating one embodiment of a system for providing a smart activity score 700. System 700 includes apparatus for providing a smart activity score 702, communication medium 704, server 706, and computing device 708.

Communication medium 704 may be implemented in a variety of forms. For example, communication medium 704 may be an Internet connection, such as a local area network (“LAN”), a wide area network (“WAN”), a fiber optic network, internet over power lines, a hard-wired connection (e.g., a bus), and the like, or any other kind of network connection. Communication medium 704 may be implemented using any combination of routers, cables, modems, switches, fiber optics, wires, radio, and the like. Communication medium 704 may be implemented using various wireless standards, such as Bluetooth, Wi-Fi, 4G LTE, etc. One of skill in the art will recognize other ways to implement communication medium 704.

Server 706 directs communications made over communications medium 704. Server 706 may be, for example, an Internet server, a router, a desktop or laptop computer, a smartphone, a tablet, a processor, a module, or the like. In one embodiment, server 706 directs communications between communications medium 704 and computing device 708. For example, server 706 may update information stored on computing device 708, or server 706 may send information to computing device 708 in real time.

Computing device 708 may take a variety of forms, such as a desktop or laptop computer, a smartphone, a tablet, a processor, a module, or the like. In addition, computing device 708 may be a processor or module embedded in a wearable sensor, a bracelet, a smart-watch, a piece of clothing, an accessory, and so on. For example, computing device 708 may be substantially similar to devices embedded in electronic capsule 200, which may be embedded in and removable from wristband 100, as illustrated in FIG. 1. Computing device 708 may communicate with other devices over communication medium 704 with or without the use of server 706. In one embodiment, computing device 708 includes apparatus 702. In various embodiments, apparatus 702 may be used to perform various processes.

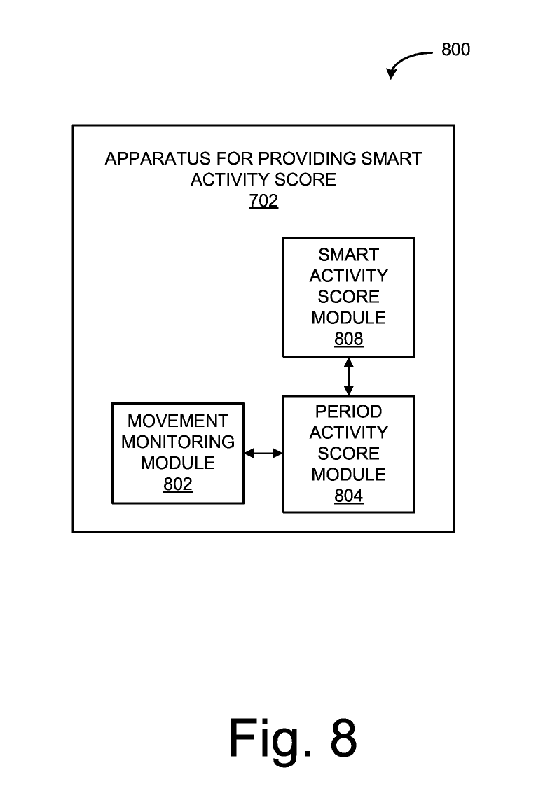

FIG. 8 is a schematic block diagram illustrating one embodiment of an apparatus for providing smart activity score 800. Apparatus 800 includes apparatus for providing a smart activity score 702 with movement monitoring module 802, period activity score module 804, and smart activity score module 808.

Movement monitoring module 802 monitors a movement to determine a metabolic loading associated with the movement during a score period. Movement monitoring module 802 will be described below in further detail with regard to various processes.

Period activity score module 804 creates and updates a period activity score based on the metabolic loading and the movement. The period activity score is created and updated for the score period. Period activity score module 804 will be described below in further detail with regard to various processes.

In one embodiment, a fatigue level module (not shown) detects a fatigue level. The fatigue level module will be described below in further detail with regard to various processes.

Smart activity score module 808 creates and updates a smart activity score by aggregating a set of period activity scores. Smart activity score module 808 will be described below in further detail with regard to various processes.

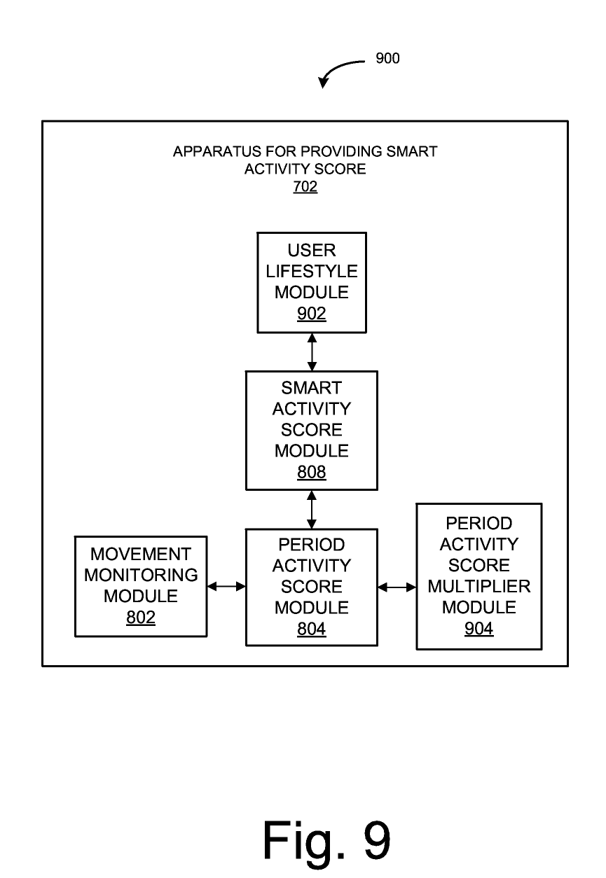

FIG. 9 is a schematic block diagram illustrating one embodiment of apparatus 900 for providing a smart activity score. Apparatus 900 includes apparatus for providing a smart activity score 702 with movement monitoring module 802, period activity score module 804, and smart activity score module 808. Apparatus 900 also includes user lifestyle module 902 and period activity score multiplier module 904. User lifestyle module 902 and period activity score multiplier module 904 will be described below in further detail with regard to various processes.

In various embodiments, at least one of movement monitoring module 802, period activity score module 804, smart activity score module 808, user lifestyle module 902, and period activity score multiplier module 904 is embodied in a wearable sensor, such as electronic capsule 200. Moreover, any of the modules described herein may be embodied in electronic capsule 200 or in other hardware or devices. Any of the modules described herein may connect to other modules described herein via communication medium 704.

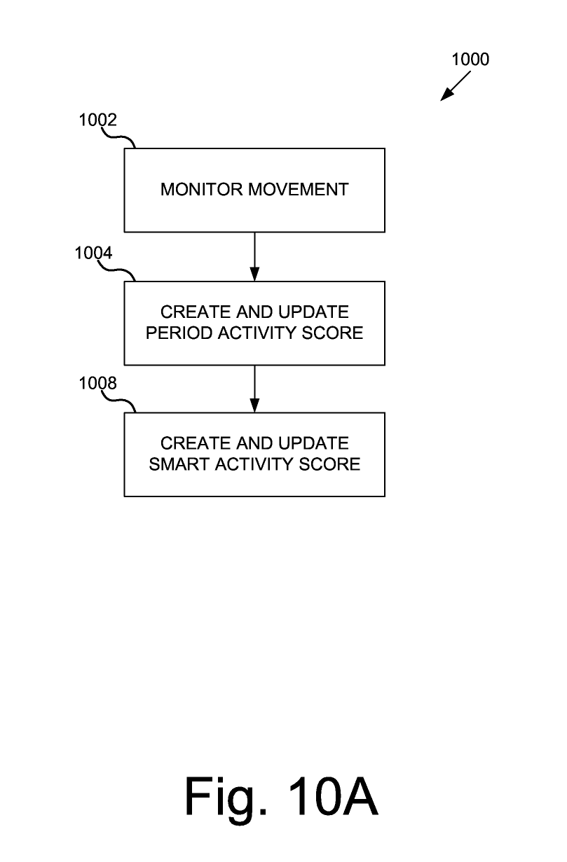

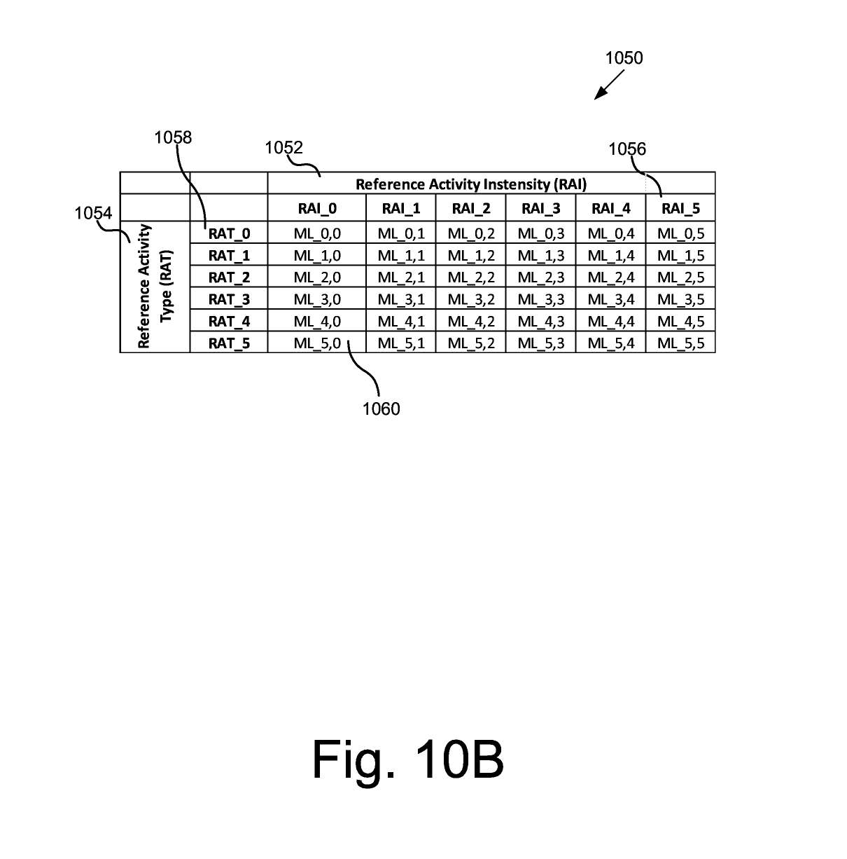

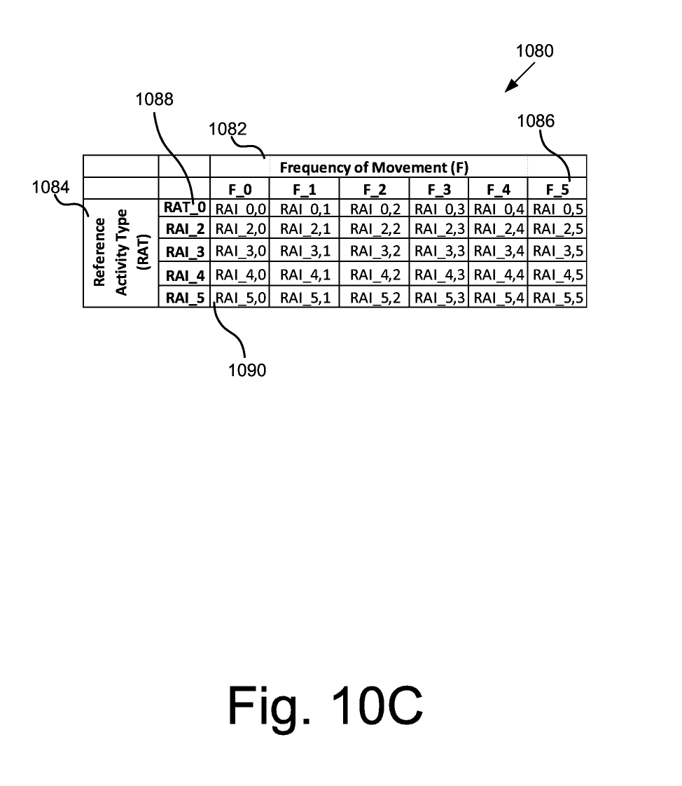

FIG. 10A is an operational flow diagram illustrating an example of a method 1000 for creating and updating a smart activity score in accordance with an embodiment of the present disclosure. The operations of method 1000 create and update a smart activity score based on a user's activity. Moreover, the operations of method 1000 take into account changes that occur in user activities as they occur in short time segments. This provides high-resolution activity monitoring. In an additional embodiment, metabolic loadings are tailored to the specific characteristics of the user. This provides for increased accuracy in tracking the user's activity levels. In one embodiment, apparatus 702, wristband 100, and electronic capsule 200 perform various operations of method 1000.

At operation 1002, method 1000 monitors a movement to determine a metabolic loading associated with the movement during a score period. In one embodiment, the metabolic loadings are determined by identifying a user activity type from a set of reference activity types and by identifying a user activity intensity from a set of reference activity intensities.

Method 1000, in one embodiment, determines a set of metabolic loadings according to information provided by a user (or user information). User information may include, for example, an individual's height, weight, age, gender, and geographic and environmental conditions. The user may provide the user information by, for example, a user interface of computing device 708, or of electronic capsule 200. Method 1000 may determine the user information based on various measurements. For example, method 1000 may determine a user's body fat content or body type. Or, for example, method 1000 may use an altimeter or GPS to determine the user's elevation, weather conditions in the user's environment, etc. In another embodiment, method 1000 obtains user information from the user indirectly. For example, method 1000 may collect user information from a social media account, from a digital profile, or the like.

In one embodiment, the user information includes a user lifestyle selected from a set of reference lifestyles. At operation 1002, method 1000 prompts the user for information about the user's lifestyle (e.g., via a user interface). Method 1000 may prompt the user to determine how active the user's lifestyle is. For example, method 1000 may prompt the user to select a user lifestyle from a set of reference lifestyles. In one embodiment, the reference lifestyles include a range of lifestyles from inactive, on one end, to highly active on the other end. So, for example, the reference lifestyles that the user may select from may include sedentary, mildly active, moderately active, and heavily active.

At operation 1002, method 1000, in one embodiment, determines the user lifestyle from the user as an initial matter. In a further embodiment, method 1000 periodically prompts the user to select a user lifestyle. In this fashion, the user lifestyle selected may be aligned with the user's actual activity level as the user's activity level varies over time. In a further embodiment, method 1000 updates the user lifestyle without intervention from the user.

In one embodiment, the metabolic loadings are numerical values and may represent a rate of calories burned per unit weight per unit time (e.g., having units of kcal per kilogram per hour). By way of example, the metabolic loadings can be represented in units of oxygen uptake (e.g., in milliliters per kilogram per minute). The metabolic loadings may also represent a ratio of the metabolic rate during activity (e.g., the metabolic rate associated with a particular activity type and/or an activity intensity) to the metabolic rate during rest. The metabolic loadings, may, for example, be represented in a metabolic table, such as metabolic table 1050 in FIG. 10B. In one embodiment, the metabolic loadings are specific to the user information. For example, a metabolic loading may increase for a heavier user, or for an increased elevation, but may decrease for a lighter user or for a decreased elevation.

At operation 1002, in one embodiment, method 1000 determines the set of metabolic loadings based on the user lifestyle, in addition to the other user information. For example, the metabolic loadings for a user with a heavily active lifestyle may differ from the metabolic loadings for a user with a sedentary lifestyle. Method 1000 may attain greater coupling between the metabolic loadings and the user's characteristics by determining the set of metabolic loadings according to the user lifestyle.

In various embodiments, a device (e.g., computing device 708) or a module (e.g., electronic capsule 200 or a module therein) stores or provides the metabolic loadings. The metabolic loadings may be maintained or provided by server 706 or over communication medium 704. In one embodiment, a system administrator provides the metabolic loading based on a survey, publicly available data, scientifically determined data, compilation of user data, or any other source of data. Operation 1002, in various embodiments, is performed by movement monitoring module 802. In various embodiments, movement monitoring module 802 includes a metabolic loading module and a metabolic table module that determine the metabolic loading associated with the movement.

At operation 1002, method 1000, in one embodiment, maintains a metabolic table based on the user information. For example, the metabolic loadings in the metabolic table may be based on the user information from the user. In some cases, the metabolic table is maintained based on a set of standard user information, in place of or in addition to user information from the user. The standard user information may include, for example, the average fitness characteristics of all individuals being the same age as the user, the same height as the user, etc. In another embodiment, instead of maintaining the metabolic table based on standard information, if method 1000 has not obtained user information from the user, method 1000 delays maintaining the metabolic table until the user information is obtained.

As illustrated in FIG. 10B at operation 1002, in one embodiment, method 1000 maintains the metabolic table as metabolic table 1050. Metabolic table 1050 may be stored in computing device 708, for example. Metabolic table 1050 may include information such as reference activity types (RATs) 1054, reference activity intensities (RAIs) 1052, and/or metabolic loadings (MLs) 1060.

In one embodiment, RATs 1054 are arranged as rows 1058 in metabolic table 1050. Thus, each of a set of rows 1058 corresponds to different RATs 1054, and each row 1058 is designated by a row index number. For example, the first RAT row 1058 may be indexed as RAT_{—}0, the second as RAT_{—}1, and so on for as many rows as metabolic table 1050 may include.

The reference activity types may include typical activities, such as running, walking, sleeping, swimming, bicycling, skiing, surfing, resting, working, and so on. The reference activity types may also include a catch-all category, for example, general exercise. The reference activity types may also include atypical activities, such as skydiving, SCUBA diving, and gymnastics. In one embodiment, a user defines a user-defined activity by programming computing device 708 (e.g., by an interface on electronic capsule 200) with information about the user-defined activity, such as pattern of movement, frequency of pattern, and intensity of movement. The typical reference activities may be provided, for example, by metabolic table 1050.

In one embodiment, reference activity intensities 1052 are arranged as columns 1056 in metabolic table 1050, and metabolic table 1050 includes columns 1056, each corresponding to different RAIs 1052. Each column 1056 is designated by a different column index number. For example, the first RAI column 1056 may be indexed as RAI_{—}0, the second as RAI_{—}1 and so on for as many columns as metabolic table 1050 may include.

The reference activity intensities, in one illustrative case, include a numeric scale. For example, the reference activity intensities may include numbers ranging from one to ten (representing increasing activity intensity). The reference activities may also be represented as a range of letters, colors, and the like. The reference activity intensities may be associated with the vigorousness of an activity. In other embodiments, the reference activity intensity are represented by ranges of heart rates or breathing rates.

In one embodiment, metabolic table 1050 includes metabolic loadings, such as metabolic loading 1060. Each metabolic loading 1060 corresponds to a reference activity type 1058 of the reference activity types 1054 and a reference activity intensity 1056 of the reference activity intensities 1052. Each metabolic loading 1060 may be identified by a unique combination of reference activity type 1054 and reference activity intensity 1052. For example, in the column and row arrangement discussed above, one of the reference activity types 1054 of a series of rows 1058 of reference activity types, and one of the reference activity intensities 1052 of a series of columns 1056 of reference activity intensities may correspond to a particular metabolic loading 1060. In such an arrangement, each metabolic loading 1060 may be identifiable by only one combination of reference activity type 1058 and reference activity intensity 1056.

This concept is illustrated in FIG. 10B. As shown, each metabolic loading 1060 is be designated using a two-dimensional index, with the first index dimension corresponding to the row 1058 number and the second index dimension corresponding to the column 1056 number of the metabolic loading 1060. For example, in FIG. 10B, ML_{—}2,3 has a first dimension index of 2 and a second dimension index of 3. ML_{—}2,3 corresponds to the row 1058 for RAT_{—}2 and the column 1056 for RAI_{—}3. Any combination of RAT_M and RAIN may identify a corresponding ML_M,N in metabolic table 1050, where M is any number corresponding to a row 1058 number in metabolic table 1050 and N is any number corresponding to a column 1056 number in metabolic table 1050. For example, the reference activity type RAT_{—}3 may be “surfing,” and the reference activity intensity RAI_{—}3 may be “4.” This combination in the metabolic table 1050 corresponds to metabolic loading 1060 ML_{—}3,3, which may, for example, represent 5.0 kcal/kg/hour (a typical value for surfing). In various embodiments, operation 1002 is performed by movement monitoring module 802. In some embodiments, operation 1002 is performed by a metabolic table module.

Referring again to operation 1002 of method 1000, in one embodiment, the movement is monitored by location tracking (e.g., Global Positioning Satellites (GPS), or a location tracking device connected via communication medium 704). In some instances, the general location of the user as well as specific movements of the user's body are monitored. For example, method 1000 may monitor the movement of the user's leg in x, y, and z directions (e.g., by an accelerometer or gyroscope). In one embodiment, method 1000 receives an instruction regarding which body part is being monitored. For example, method 1000 may receive an instruction that the movement of a user's wrist, ankle, head, or torso is being monitored.

Method 1000, in various embodiments, monitors the movement of the user and determines a pattern of the movement (pattern). For example, method 1000 may detect the pattern by an accelerometer or gyroscope. The pattern may be a repetition of a motion or a similar motion monitored at operation 1002. In one embodiment, the pattern is a geometric shape (e.g., a circle, line, oval) of repeated motion that is monitored. In some cases, the repetition of a motion in a geometric shape is not repeated consistently over time, but is maintained for a substantial proportion of the repetitions of movement. For example, one occurrence of elliptical motion in a repetitive occurrence (or pattern) of ten circular motions may be monitored and determined to be a pattern of circular motion.

In further embodiments, the geometric shape of the pattern of movement is a three dimensional (3-D) shape. For example, the pattern of movement associated with the wrist of a person swimming the butterfly stroke may be monitored and analyzed into a geometric shape in three dimensions. The pattern may be complicated, but it may be described in a form that method 1000 can recognize when performing operation 1002. Such form may include computer code that describes the spatial relationship of a set of points, along with changes in acceleration forces that are experienced along those points as, for example, a sensor travels throughout the pattern.

At operation 1002, in some instances, monitoring the pattern includes monitoring the frequency with which the pattern is repeated (or pattern frequency). The pattern frequency may be derived from a repetition period of the pattern (or pattern repetition period). The pattern repetition period may be the length of time elapsing from when a device or sensor passes through a certain point in a pattern and when the device or sensor returns to that point when the pattern is repeated. For example, the sensor may be at point x, y, z at time t_{—}0. The device may then move along the trajectory of the pattern, eventually returning to point x, y, z at time t_{—}1. The pattern repetition period would be the difference between t_{—}1 and t_{—}0 (e.g., measured in seconds). For example, method 1000 may determine that the pattern is a circle and that the circle pattern repetition period is one second. The pattern frequency may be the inverse of the pattern repetition period, and may have units of cycles per second. When the pattern repetition period is, for example, two seconds, the pattern frequency would be 0.5 cycles per second.

In various embodiments, monitoring the movement at operation 1002 includes monitoring the velocity at which the user is moving (or the user velocity). For example, the user velocity may have units of kilometers per hour. Method 1000, in one embodiment, monitors the user's location information to determine user velocity. Method 1000 may do this by GPS, through communication medium 704, and so on. The user velocity may be distinguished from the speed of the pattern (or pattern speed). For example, the user may be running at a user velocity 10 km/hour, but the pattern speed of the user's wrist may be 20 km/hour at a given point (e.g., as the wrist moves from behind the user to in front of the user). At operation 1002, the pattern speed may be monitored using, for example, an accelerometer or gyroscope.

At operation 1002, in one embodiment, method 1000 monitors the user's altitude. This may be done, for example, using an altimeter. Method 1000 may do this using other means, such as use location information, information entered by the user, etc. In another embodiment, at operation 1002, method 1000 monitors an impact the user has with an object. For example, method 1000 may monitor the impact of the user's feet with ground. Method 1000 may do this using, for example, an accelerometer or gyroscope.

In various embodiments, method 1000 measures the ambient temperature. For example, method 1000 may associate a group of reference activity types with bands of ambient temperature. For example, when the ambient temperature is zero degrees Celsius, activities such as skiing, sledding, and ice climbing are appropriate selections for reference activity types, whereas surfing, swimming, and beach volleyball may be inappropriate. In further embodiments, the humidity may be measured (e.g., by a hygrometer). In further embodiments, at operation 1002, method 1000 measures the pattern duration, that is, the length of time for which particular movement pattern is sustained.

Method 1000, in some cases, performs operation 1002, monitoring the movement, by using a sensor configured to be attached to a user's body. Such sensors may include a gyroscope or accelerometer to detect movement, and a heart-rate sensor, each of which may be embedded in a wristband that a user can wear on the user's wrist or ankle, such as wristband 100. Additionally, various modules and sensors that may be used to perform operation 1002 may be embedded in an electronic capsule, such as electronic capsule 200. In various embodiments, operation 1002 is performed by movement monitoring module 802.

In one embodiment, operation 1002 involves determining the user activity type from the set of reference activity types. Once detected, the pattern may be used to determine the user activity type from a set of reference activity types. In one illustrative instance, each reference activity type is associated with a reference activity type pattern. The user activity type may be determined to be the reference activity type that has a reference activity type pattern that matches the pattern measured at operation 1002. In one embodiment, the pattern that matches the reference activity type pattern will not be an exact match, but will be substantially similar.

The patterns, in other embodiments, will not even be substantially similar, but method 1000 will determine that the patterns match because they are the most similar of any patterns available. For example, the reference activity type may be determined such that the difference between the pattern of movement corresponding to this reference activity type and the pattern of movement is less than a predetermined range or ratio. In one embodiment, the pattern is looked up (for a match) in a reference activity type library. The reference activity type library may be included in the metabolic table. For example, the reference type library may include rows in a table such as the RAT rows 1058.

In further embodiments, operation 1002 involves using the pattern frequency to determine the user activity type from the set of reference activity types. For example, several reference activity types may be associated with similar patterns (e.g., because the wrist moves in a similar pattern when running versus walking). Method 1000 may measure a pattern and not be able to determine whether the corresponding user activity type is walking or running. Method 1000 may use the pattern frequency to determine the activity type in such an example because the pattern frequency for running may be higher than the pattern frequency for walking.

Operation 1002 involves, in one embodiment, using additional information to determine the activity type of the user. For example, the pattern for walking may be similar to the pattern for running. Method 1000 may associate the reference activity of running with higher user velocities and may associate the reference activity of walking with lower user velocities. Method 1000 may use the velocity measured at operation 1002 to determine between two reference activity types having similar patterns.

In another embodiment, operation 1002 involves monitoring the impact the user has with the ground, and determine that, because the impact is larger, the activity type is running rather than walking. Moreover, if there is no impact, method 1000 may determine that the activity type is cycling (or other activity where there is no impact). In another embodiment, method 1000 uses the humidity measurement to determine whether the activity is a water sport, that is, whether the activity is being performed in the water. Method 1000 may narrow the reference activity types to those that are performed in the water, from which narrowed set of reference activity types the user activity type may be determined. In another embodiment, method 1000 uses the temperature measurement to determine the activity type.

Operation 1002, in another case, entails instructing the user to confirm the user activity type. For example, a user interface may be provided such that the user may confirm whether a displayed user activity type is correct. In another embodiment, a user interface is provided such that the user may select the user activity type from a group of activity types.

In further embodiments, at operation 1002, method 1000 determines a statistical likelihood for of choices for user activity type and provide the possible user activity types in such a sequence that the most likely user activity type is listed first (and then in descending order of likelihood). For example, method 1000 may detect a pattern of movement and determine that, based on the pattern, the pattern frequency, the temperature, and so on, there is an 80% chance the user activity type is running, a 15% chance the user activity type is walking, and a 5% chance the user activity is dancing. Method 1000 may then, via a user interface, list these possible user activities such that the user may select the activity type the user is performing. In various embodiments, portions of operation 1002 are performed by a metabolic loading module.

At operation 1002, in one embodiment, method 1000 determines the user activity intensity from a set of reference activity intensities. Method 1000 may determine the user activity intensity in a variety of ways. In one embodiment, method 1000 associates the repetition period (or pattern frequency) and user activity type (UAT) with a reference activity intensity library to determine the user activity intensity that corresponds to a reference activity intensity. FIG. 10C illustrates one embodiment whereby this aspect of operation 1002 is accomplished, including reference activity intensity library 1080. Library 1080 is organized by rows 1088 of reference activity types 1084 and columns 1086 of pattern frequencies 1082. In FIG. 10C, library 1080 is implemented in a table. Library 1080 may, however, be implemented other ways.

In one embodiment, at operation 1002, method 1000 determines that, for user activity type 1084 UAT_{—}0 performed at pattern frequency 1082 F_{—}0, the reference activity intensity 1090 is RAI_{—}0,0. For example, method 1000 may determine that UAT 1084 corresponds to the reference activity type for running. Method 1000 may also determine a pattern frequency 1082 of 0.5 cycles per second for the user activity type. Reference activity intensity library 1080 may determine, at operation 1002, that the user activity type 1084 of running at a pattern frequency 1082 of 0.5 cycles per second corresponds to a reference activity intensity 1090 of five on a scale of ten. In another embodiment, the reference activity intensity 1090 is independent of the activity type. For example, method 1000 may determine that the repetition period is five seconds, and that this corresponds to an intensity level of two on a scale of ten.

Reference activity intensity library 1080, in one embodiment, is included in metabolic table 1050. In some cases, the measured repetition period (or pattern frequency) does not correspond exactly to a repetition period for a reference activity intensity in metabolic table 1050. In such an example, the correspondence may be a best-match fit, or may be a fit within a tolerance. Such a tolerance may be defined by the user or by a system administrator, for example.

In various embodiments, operation 1002 involves supplementing the measurement of pattern frequency to help determine the user activity intensity from the reference activity intensities. For example, if the user activity type is skiing, it may be difficult to determine the user activity intensity because the pattern frequency may be erratic or otherwise immeasurable. In such an example, method 1000 may monitor the user velocity, the user's heart rate, and other indicators (e.g., breathing rate) to determine how hard the user is working during the activity. For example, higher heart rate may indicate lower activity intensity. In a further embodiment, the reference activity intensity are associated with a pattern speed (i.e., the speed or velocity at which the sensor is progressing through the pattern). A higher pattern speed may correspond to a higher user activity intensity.

Method 1000, in some instances, performs operation 1002 to determine the user activity type and the user activity intensity by using a sensor configured to be attached to the user's body. Such sensors may include, for example, a gyroscope or accelerometer to detect movement, and a heart-rate sensor, each of which may be embedded in a wristband that a user can wear on the user's wrist or ankle, such as wristband 100. Additionally, various sensors and modules that may be used to preform operation 1002 may be embedded in electronic capsule 200. In various embodiments, operation 1002 is performed by movement monitoring module 802.

Referring again to FIG. 10A, operation 1004 includes creating and updating a period activity score based on the metabolic loading and the movement. The period activity score is created and updated for the score period. In one embodiment, method 1000 (e.g., at operation 1002) determines a duration of the activity type at a particular activity intensity (e.g., in seconds, minutes, or hours). Method 1000 may create and update the period activity score by multiplying the metabolic loading by the duration of the user activity type at a particular user activity intensity. If the user activity intensity changes, method 1000 may multiply the new metabolic loading (associated with the new user activity intensity) by the duration of the user activity type at the new user activity intensity. Accordingly, in one embodiment, the period activity score is represented as a numerical value.

Additionally, at operation 1004, method 1000 creates and updates the period activity score based on score periods. In one embodiment, at operation 1002, monitoring the movement includes determining, during a score period, the metabolic loading associated with the movement. Score periods may include segments of time. For example, a score period may be ten seconds. For the illustrative score period of ten seconds, each twenty-four day would include 8,640 score periods. In one embodiment, method 1000 monitors the user's movement (e.g., at operation 1002) to determine a user activity type, a user activity intensity, and a corresponding metabolic loading during each score period. Method 1000 may then calculate the period activity score for that score period. As the movement changes over time, the varying characteristics of the movement are captured by the score periods.

In one embodiment, operation 1004 includes creating and updating a set of periodic activity scores. Each period activity is based on the movement monitored during a set of score periods, and each period activity score is associated with a particular score period of the set of score periods. In one embodiment, creating and updating the smart activity score includes aggregating a set of period activity scores. The smart activity score may represent a running sum total of the period activity scores.

Operation 1004, in one embodiment, includes applying a score period multiplier to the score period to create an adjusted period activity score. In such an embodiment, the smart activity score includes an aggregation of adjusted period activity scores. For example, method 1000 may introduce score period multipliers associated with certain score periods, such that the certain score periods contribute more or less to the period activity score than other score periods during which the same movement is monitored. For example, if the user is performing a sustained activity, method 1000 may apply a score period multiplier to the score periods that occur during the sustained activity. By contrast, method 1000 may not apply a multiplier to score periods that are part of intermittent, rather than sustained, activity. As a result of the score period multiplier, the user's sustained activity may contribute more to the metabolic activity score than the user's intermittent activity. The score period multiplier may allow method 1000 to account for the increased demand of sustained, continuous activity relative to intermittent activity.

In one embodiment, the score period multiplier is directly proportional to the number of continuous score periods over which a type and intensity of the movement is maintained. The adjusted period activity score may be greater than or less than the period activity score, depending on the score period multiplier. For example, for intermittent activity, the score period multiplier may be less than 1.0, whereas for continuous, sustained activity, the score period multiplier may be greater than 1.0.

The score period multiplier, in a further embodiment, is directly proportional to the smart activity score for the current measuring period. For example, a user who already has a smart activity score of 2,000 will receive a greater period activity score for going running that a user who has a smart activity score of 1,000. In this way, method 1000 may allocate greater points for highly active days relative to moderately active days.

In one embodiment, operation 1004 entails decreasing the smart activity score when the user consumes calories. For example, if the user goes running and generates an activity score of 1,000 as a result, but then the user consumes calories, method 1000 may decrease the smart activity score by 200 points, or any number of points. The decrease in the number of points may be proportional to the number of calories consumed. In other embodiments, method 1000 obtains information about specific aspects of the user's diet, and awards metabolic activity score points for healthy eating (e.g., fiber) and subtracts points for unhealthy eating (e.g., excessive fat consumption).

Method 1000, in one embodiment, pushes the user to work harder, or not as hard, depending on the user lifestyle. Method 1000 may do this, for example by adjusting the metabolic loadings based on the user lifestyle. For example, a user with a highly active lifestyle may be associated with metabolic loadings that result in a lower metabolic activity score when compared to a user with a less active lifestyle performing the same movements. This results in method 1000 requiring the more active user to, for example, work (or perform movement) at a higher activity intensity or for a longer duration to achieve the same metabolic activity score as the less active user participating in the same activity type (or movements).

At operation 1004, in one embodiment, the smart activity score is reset every twenty-four hours. Method 1000 may continually increment and decrement the smart activity score throughout a measuring period, but may reset the smart activity score to a value (e.g., zero) at the end of twenty-four hours. The smart activity score may be reset after any given length of time (or measuring period). The smart activity score may be continually updated over the period of, for example, one week, or one month.

In one embodiment, method 1000 determines that, because the smart activity score was greater than a certain amount for the measuring period, the smart activity score should be reset to a number greater than zero. As such, the user effectively receives a credit for a particularly active day, allowing the user to be less active the next day without receiving a lower smart activity score for the next day. In a further embodiment, method 1000 determines that, because the smart activity score was less than a predetermined value for the measuring period, the smart activity score should be reset to a value less than zero. The user effectively receives a penalty for that day, and would have to make up for a particularly inactive, or overly consumptive day by increasing the user's activity levels the next day. In various embodiments, operation 1004 is performed by smart activity score module 808.

Method 1000, in one embodiment, includes the operation of detecting a fatigue level. In one embodiment, recovery is a function of the fatigue level. In one embodiment, the fatigue level is the fatigue level of the user. Method 1000 may detect the fatigue level in various ways. In one embodiment, method 1000 detects the fatigue level by measuring a heart rate variability (HRV) of a user using logic circuits 240 (discussed above in reference in to FIG. 1). For example, when the HRV is more consistent (i.e., steady, consistent amount of time between heartbeats), the fatigue level may be higher. In other words, the body is less fresh and well-rested. When HRV is more sporadic (i.e., amount of time between heartbeats varies largely), the fatigue level may be lower.

Method 1000 may measure HRV in a number of ways (discussed above in reference in to FIG. 1). For example, in one embodiment, method 1000 measures HRV using the combination of wrist biosensor 210 and finger biosensor 220. Wrist biosensor 210 may, for example, measure the heartbeat in the wrist of one arm while finger sensor 220 measures the heartbeat in a finger of the hand of the other arm. This combination allows the sensors, which in one embodiment may be conductive, to measure an electrical potential through the body. In one embodiment, information about the electrical potential provides cardiac information (e.g., HRV, fatigue level, heart rate information, and so on) and such information is processed. In other embodiments, method 1000 measures the HRV using sensors that monitor other parts of the user's body, rather than the finger and wrist. For example, the sensor may monitor the ankle, leg, arm, or torso.

In one embodiment, method 1000 detects the fatigue level based solely on the HRV measured. In a further embodiment, the fatigue level is based on other measurements (e.g., measurements monitored at operation 1002). For example, the fatigue level may be based on the amount of sleep that is measured for the previous night, the duration and type of user activity, and the intensity of the activity that method 1000 may determine for a previous time period (e.g., exercise activity level in the last twenty-four hours). By way of example, the factors may include stress-related activities such as work and driving in traffic, which may generally cause a user to become fatigued. In some cases, method 1000 detects the fatigue level by comparing the HRV measured to a reference HRV. This reference HRV may be based on information gathered from a large number of people from the general public. In another embodiment, method 1000 determines the reference HRV based on past measurements of the user's HRV.

Method 1000, in other illustrative instances, detects the fatigue level once every twenty-four hours. This provides information about the user's fatigue level each day so that method 1000 may direct the user's activity levels accordingly. In one embodiment, the fatigue level is detected more or less often. Using the fatigue level, a user may determine whether or not an activity is necessary, the appropriate activity intensity, and the appropriate activity duration. For example, in deciding whether to go on a run, or how long to run, the user may want to use method 1000 to assess the user's current fatigue level. Then the user may, for example, run for a shorter time if the user is more fatigued, or for a longer time if the user is less fatigued.

Referring again to FIG. 10A, at operation 1008, method 1000 creates and updates a smart activity score by aggregating a set of period activity scores. In one embodiment, method 1000 may create the smart activity score by increasing or decreasing the period activity scores according to the fatigue level. The fatigue level is represented as a numerical value. In one embodiment, the fatigue level is represented as a relative value, for example, as a current fatigue level relative to an average fatigue level for the user. Method 1000 may use this relative value to scale, increment, or decrement the period activity scores to create the smart activity score. The smart activity score may account not only for the movement of the user, but also for the recovery state, or fatigue level, of the user.

In one embodiment, the smart activity score is tuned to the user's fatigue level, and provides information about whether the user is in his or her peak recovery zone. In such an embodiment, by modifying the period activity score based on the fatigue level, operation 1008 pushes the user to exercise more or less vigorously (according to the movement's activity type, intensity, or duration) based on various factors that may affect the user's body. Such factors may include sleep amount, stress levels, general lifestyle and health levels, past workout routines, and recent exercise levels. Operation 1008, in one instance, involves updating the smart activity score as the period activity scores are aggregated over time (e.g., according to score periods). In one embodiment, operation 1008 performs this updating in real time. In other cases, the updating is delayed for a period of time.

The smart activity score, in one embodiment, is associated with a measuring period. The smart activity score may be incremented or decremented throughout the measuring period according to the user's movement, including the user activity types and the user activity intensities. In one embodiment, the smart activity score is reset at the end of the measuring period. For example, the smart activity score may be reset to zero or a number other than zero. In another embodiment, the smart activity score is associated with a measuring period that begins when method 1000 detects the fatigue level. In one embodiment, the measuring period is twenty-four hours. However, the measuring period may be any amount of time. For example, the measuring period may be one week, one month, and so on, or may be associated with a training schedule for a race or other event. In various embodiments, operation 1008 is performed by smart activity score module 808.

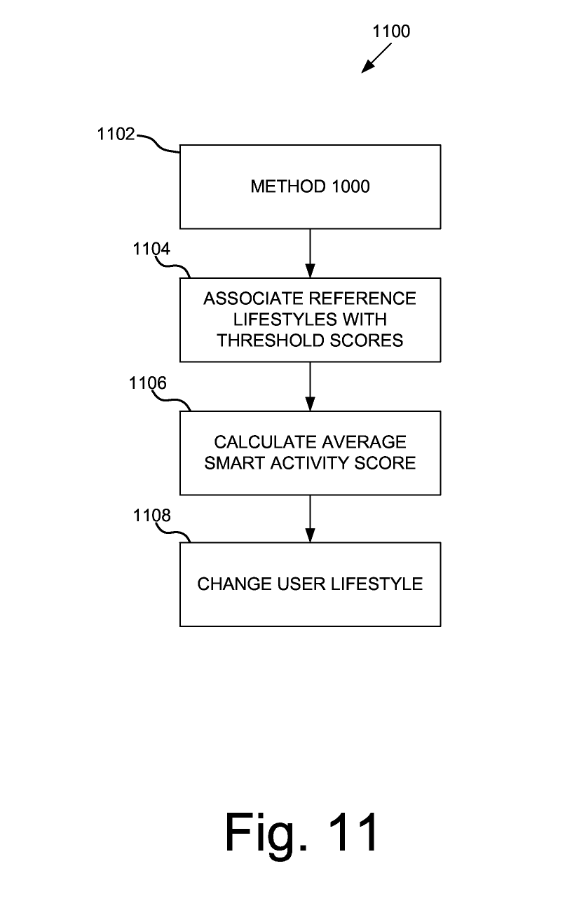

FIG. 11 is an exemplary operational flow diagram illustrating one embodiment of method 1100 for creating and updating a smart activity score. Method 1100 associates reference lifestyles with threshold scores (e.g., at operation 1104), calculates an average smart activity score (e.g., at operation 1106), and changes a user lifestyle (e.g., at operation 1108). Method 1100 may also include all the operations of method 1000, in some cases.

At operation 1104, method 1100 associates each reference lifestyle with a lower threshold score and an upper threshold score. In one embodiment, the lower threshold score and the upper threshold score are numerical values. For example, method 1100 may associate the sedentary reference lifestyle with a lower threshold score of 1,000 and an upper threshold score of 2,000. In addition, method 1100 may associate the mildly active reference lifestyle with a lower threshold score of 2,001 and an upper threshold score of 3,000. The lower threshold score and the upper threshold score associated with each reference lifestyle define a range of threshold scores. In one embodiment, no two ranges of threshold scores overlap. In various embodiments, operation 1104 is performed by user lifestyle module 902.

At operation 1106, method 1100 calculates an average smart activity score from a set of past smart activity scores. Method 1100 may calculate the average smart activity score using a mean, median, mode, or other statistical measure. In one embodiment, the average smart activity score is a range that includes a certain number of standard deviations from a mean or median smart activity score. In one instance, each past smart activity score is associated with a past measuring period. In various embodiments, operation 1106 is performed by smart activity score module 808.

At operation 1108, method 1100 changes the user lifestyle to the reference lifestyle associated with the lower threshold score not greater than the average smart activity score and the upper threshold score not less than the average smart activity score. For example, at operation 1106, method 1100 may calculate the mean smart activity score from each day over the past month to be 3,500 per day, and the user lifestyle may be mildly active. In one embodiment, the mildly active reference lifestyle has a lower and upper threshold score of 2,001 and 3,000, respectively, and the moderately active reference lifestyle has a lower and upper threshold score of 3,001 and 4,000, respectively. Method 1100, at operation 1108 (in this example), changes the user lifestyle from mildly active to moderately active because the average smart activity score is between 3,001 and 4,000 (i.e., 3,500), the range associated with moderately active. In other embodiments, method 1100 includes customizable upper and lower threshold scores for each reference lifestyle. Operation 1108, in various embodiments, is performed by smart activity score module 808 or by metabolic table module 1050.

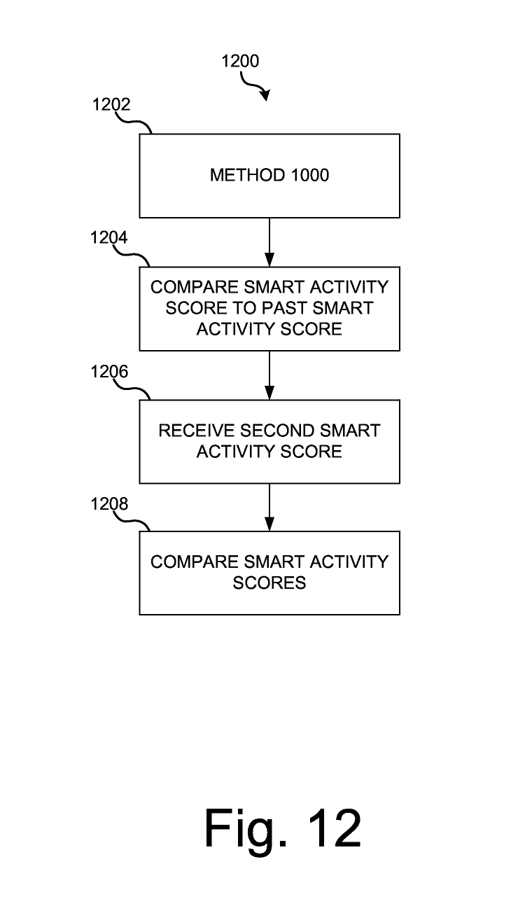

FIG. 12 is an exemplary operational flow diagram illustrating one embodiment of method 1200 for creating and updating a smart activity score. Method 1200 compares the smart activity score to a past smart activity score (e.g., at operation 1204), receives a second smart activity score (e.g., at operation 1206), and compares the smart activity score to the second smart activity score (e.g., at operation 1208).

At operation 1204, method 1200 compares the smart activity score to a past smart activity score, and the past smart activity score is associated with a past measuring period. In one embodiment, method 1200 stores smart activity scores associated with past measuring periods. Method 1200 may recall any past smart activity score and use information associated with that past smart activity score to inform the user's current activity.

At operation 1204, in one embodiment, method 1200 compares the smart activity score to the smart activity score from the past measuring period by providing a simple numerical readout of both scores (e.g., side by side). In one embodiment, method 1200 presents information about the time of day associated with the past smart activity score. For example, method 1200 indicates that the past smart activity score was at a particular level at a particular time of day. For example, if the current time is 2:00 PM, method 1200 may present the information that on the past day of Oct. 12, 2013, the past smart activity score was 1,200 at 2:00 PM. This may inform the user of how the user's current smart activity score is progressing throughout the measuring period in relation to the past smart activity score.

In another embodiment, operation 1204 entails displaying a graph (e.g., a line or bar graph) of the past smart activity score as a function of time in the past measuring period (e.g., activity score on the y-axis and time on the x-axis). Method 1200 may overlay that graph with a graph of the current smart activity score as a function of time over the current measuring period. This may inform the user of the progress of the current measuring period's activity in relation to the past measuring period's activity. One of ordinary skill in the art will appreciate other ways that method 1200 may compare the smart activity scores at operation 1204. In another embodiment, method 1200 compares the smart activity score to multiple past smart activity scores associated with past measuring periods. The display of past or current smart activity scores, in a further embodiment, is broken down by amount contributed per score period. In various embodiments, operation 1204 is performed by smart activity score module 808.

At operation 1206, method 1200 receives a second smart activity score from a second user. Method 1200 may receive the second smart activity score in a number of ways. For example, method 1200 may receive the second smart activity score via communication medium 704.

The second smart activity score may be created and updated at operation 1206 in a manner substantially similar to the creating and updating of the smart activity score at operation 1008. In one embodiment, the second smart activity score represents the modified version of a second smart activity score that is modified or adjusted according to a fatigue level of the second user. In another embodiment, the second activity score may be modified by the period activity score multiplier of the second user. The second user may be any user other than the user. For example, the second user may be a friend or associate of the first user. In various embodiments, operation 1206 is performed by smart activity score module 808.

At operation 1208, method 1200 compares the smart activity score to the second smart activity score. Method 1200 may compare the smart activity score to the second smart activity score in many of the same ways that method 1200 may compare the smart activity score to the past smart activity score (e.g., at operation 1204). Method 1200 may compare the two scores using overlaid graphs or other visual displays, by using side-by-side numbers, and the like. In one example, this comparison allows the user to compare the user's daily activity level to the daily activity level of another user. In another example, both users' activity levels are tuned to each user's respective fatigue level. The measuring period, however, for the smart activity score and the second smart activity score may be different. In one instance, method 1200 takes into account possible different measuring periods for the two smart activity scores, and normalizes the scores to account for this difference. For example, if the second user is on the East Coast, and the user is on the West Coast, method 1200 may adjust the smart activity score comparison to account for this difference. In various embodiments, operation 1208 is performed by smart activity score module 808.

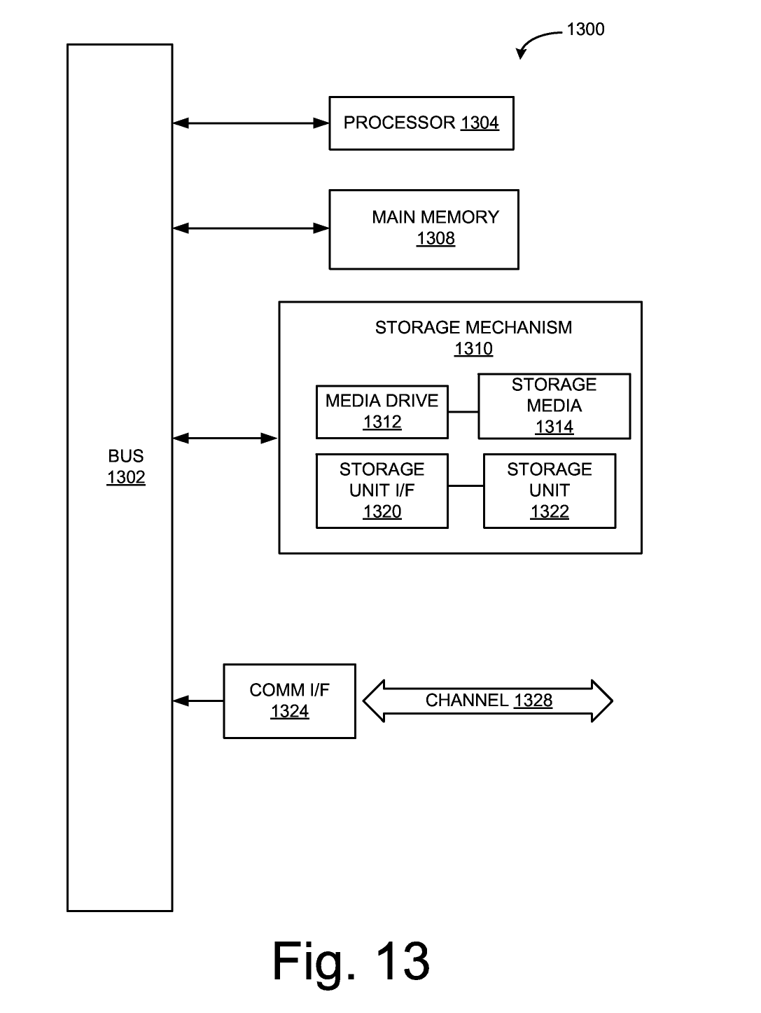

FIG. 13 illustrates an example computing module that may be used to implement various features of the systems and methods disclosed herein. In one embodiment, the computing module includes a processor and a set of computer programs residing on the processor. The set of computer programs is stored on a non-transitory computer readable medium having computer executable program code embodied thereon. The computer executable code is configured to monitor a movement to determine a metabolic loading associated with the movement during a score period. The computer executable code is configured to create and update a period activity score based on the metabolic loading and the movement during the score period. The period activity score is created and updated for the score period. The computer executable code is configured to create and update a smart activity score by aggregating a set of period activity scores.

The example computing module may be used to implement these various features in a variety of ways, as described above with reference to the methods illustrated in FIGS. 10A, 10B, 10C, 11, and 12, and as will be appreciated by one of ordinary skill in the art.

As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 13. Various embodiments are described in terms of this example-computing module 1300. After reading this description, it will become apparent to a person skilled in the relevant art how to implement the application using other computing modules or architectures.

Referring now to FIG. 13, computing module 1300 may represent, for example, computing or processing capabilities found within desktop, laptop, notebook, and tablet computers; hand-held computing devices (tablets, PDA's, smart phones, cell phones, palmtops, smart-watches, smart-glasses etc.); mainframes, supercomputers, workstations or servers; or any other type of special-purpose or general-purpose computing devices as may be desirable or appropriate for a given application or environment. Computing module 1300 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 1300 might include, for example, one or more processors, controllers, control modules, or other processing devices, such as a processor 1304. Processor 1304 might be implemented using a general-purpose or special-purpose processing engine such as, for example, a microprocessor, controller, or other control logic. In the illustrated example, processor 1304 is connected to a bus 1302, although any communication medium can be used to facilitate interaction with other components of computing module 1300 or to communicate externally.

Computing module 1300 might also include one or more memory modules, simply referred to herein as main memory 1308. For example, preferably random access memory (RAM) or other dynamic memory, might be used for storing information and instructions to be executed by processor 1304. Main memory 1308 might also be used for storing temporary variables or other intermediate information during execution of instructions to be executed by processor 1304. Computing module 1300 might likewise include a read only memory (“ROM”) or other static storage device coupled to bus 1302 for storing static information and instructions for processor 1304.

The computing module 1300 might also include one or more various forms of information storage mechanism 1310, which might include, for example, a media drive 1312 and a storage unit interface 1320. The media drive 1312 might include a drive or other mechanism to support fixed or removable storage media 1314. For example, a hard disk drive, a solid state drive, a magnetic tape drive, an optical disk drive, a CD or DVD drive (R or RW), or other removable or fixed media drive might be provided. Accordingly, storage media 1314 might include, for example, a hard disk, a solid state drive, magnetic tape, cartridge, optical disk, a CD or DVD, or other fixed or removable medium that is read by, written to or accessed by media drive 1312. As these examples illustrate, the storage media 1314 can include a computer usable storage medium having stored therein computer software or data.

In alternative embodiments, information storage mechanism 1310 might include other similar instrumentalities for allowing computer programs or other instructions or data to be loaded into computing module 1300. Such instrumentalities might include, for example, a fixed or removable storage unit 1322 and a storage interface 1320. Examples of such storage units 1322 and storage interfaces 1320 can include a program cartridge and cartridge interface, a removable memory (for example, a flash memory or other removable memory module) and memory slot, a PCMCIA slot and card, and other fixed or removable storage units 1322 and storage interfaces 1320 that allow software and data to be transferred from the storage unit 1322 to computing module 1300.

Computing module 1300 might also include a communications interface 1324. Communications interface 1324 might be used to allow software and data to be transferred between computing module 1300 and external devices. Examples of communications interface 1324 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 1324 might typically be carried on signals, which can be electronic, electromagnetic (which includes optical) or other signals capable of being exchanged by a given communications interface 1324. These signals might be provided to communications interface 1324 via a channel 1328. This channel 1328 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 1308, storage unit 1320, media 1314, and channel 1328. These and other various forms of computer program media or computer usable media may be involved in carrying one or more sequences of one or more instructions to a processing device for execution. Such instructions embodied on the medium are generally referred to as “computer program code” or a “computer program product” (which may be grouped in the form of computer programs or other groupings). When executed, such instructions might enable the computing module 1300 to perform features or functions of the present application as discussed herein.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

What is claimed is:

1. An apparatus for providing a smart activity score, the apparatus comprising:
a movement monitoring module that monitors a movement to determine a metabolic loading associated with the movement during a score period;
a period activity score module that creates and updates a period activity score based on the metabolic loading and the movement, the period activity score created and updated for the score period; and
a smart activity score module that creates and updates a smart activity score by aggregating a set of period activity scores.

2. The apparatus of claim 1, wherein the metabolic loading is determined from a set of metabolic loadings, each metabolic loading determined according to user information from a user.

3. The apparatus of claim 2,
wherein the smart activity score is associated with a measuring period; and
wherein the smart activity score module calculates an average smart activity score from a set of past smart activity scores, each past smart activity score associated with a past measuring period.

4. The apparatus of claim 3,
wherein the user information comprises a user lifestyle selected from a set of reference lifestyles; and
wherein the apparatus for providing the smart activity score further comprises a user lifestyle module that:
associates each reference lifestyle with a lower threshold score and an upper threshold score, wherein the lower threshold score and the upper threshold score associated with each reference lifestyle define a range or scores, and wherein no two ranges of scores overlap; and
changes the user lifestyle to the reference lifestyle associated with the lower threshold score not greater than the average smart activity score and the upper threshold score not less than the average smart activity score.

5. The apparatus of claim 1, further comprising:
a period activity score multiplier module that applies a period activity score multiplier to the period activity score to create an adjusted period activity score;
wherein the smart activity score comprises an aggregation of adjusted period activity scores.

6. The apparatus of claim 5, wherein the period activity score multiplier is directly proportional to the number of continuous score periods over which a user activity type of the movement and a user activity intensity of the movement is maintained.

7. The apparatus of claim 5, wherein the period activity score multiplier is directly proportional to smart activity score for the current measuring period.

8. The apparatus of claim 1, wherein the score period is ten seconds.

9. The apparatus of claim 1, wherein at least one of the movement monitoring module, the period activity score module, and the smart activity score module is embodied in a sensor configured to be attached to the body of a user.

10. A method for providing a smart activity score, the method comprising:
monitoring a movement to determine a metabolic loading associated with the movement during a score period;
creating and updating a period activity score based on the metabolic loading and the movement, the period activity score created and updated for the score period; and
creating and updating a smart activity score by aggregating a set of period activity scores.

11. The method of claim 10,
wherein the metabolic loading is determined from a set of metabolic loadings, each metabolic loading determined according to user information from a user;
wherein the user information comprises a user lifestyle selected from a set of reference lifestyles; and
wherein the step of determining the set of metabolic loadings is based on the user lifestyle.

12. The method of claim 11, further comprising:
associating each reference lifestyle with a lower threshold score and an upper threshold score, wherein the lower threshold score and the upper threshold score associated with each reference lifestyle define a range of scores, and wherein no two ranges of scores overlap;
calculating an average smart activity score from a set of past smart activity scores, each past smart activity score associated with a past measuring period; and
changing the user lifestyle to the reference lifestyle associated with the lower threshold score not greater than the average smart activity score and the upper threshold score not less than the average smart activity score.

13. The method of claim 10, further comprising comparing the smart activity score to a past smart activity score, the smart activity score associated with a measuring period, the past smart activity score associated with a past measuring period.

14. The method of claim 10, further comprising:
receiving a second smart activity score from a second user; and
comparing the smart activity score to the second smart activity score.

15. The method of claim 10, wherein the step of creating and updating the smart activity score further comprises:
applying a score period multiplier to the score period to create an adjusted period activity score;
wherein the smart activity score comprises an aggregation of adjusted period activity scores.

16. The method of claim 15, wherein the score period multiplier is directly proportional to the number of continuous score periods over which a user activity type of the movement and a user activity intensity of the movement is maintained.

17. The method of claim 10, wherein the score period multiplier is directly proportional to the smart activity score for the current measuring period.

18. The method of claim 10, wherein the score period is ten seconds.

19. The method of claim 10, wherein at least one of the steps of monitoring the movement, creating and updating the period activity score, and creating and updating the smart activity score comprises using a sensor configured to be attached to the body of the user.

20. A system for providing a smart activity score, the system comprising:
a processor; and
at least one computer program residing on the processor;
wherein the computer program is stored on a non-transitory computer readable medium having computer executable program code embodied thereon, the computer executable program code configured to:
monitor a movement to determine a metabolic loading associated with the movement during a score period;
create and update a period activity score based on the metabolic loading and the movement during the score period;
create and update a smart activity score by aggregating a set of period activity scores.
